(19) United States
(12) Patent Application Publication
(10) Pub. No.: US 2004/0259138 A1
(43) Pub. Date: Dec. 23, 2004

(54) Method for printing biomolecules

(75) Inventor: Holger Klapproth, Freiburg (DE)

Correspondence Address: GRIFFIN & SZIPL, PC, SUITE PH-1, 2300 NINTH STREET, SOUTH, ARLINGTON, VA, 22204, US

(73) Assignee: Micronas Holding GmbH, Freiburg (DE)

(21) Appl. No.: 10/845,590

(22) Filed: May 14, 2004

(30) Foreign Application Priority Data
May 14, 2003 (DE) 103 21 809.2

(51) Int. Cl. C12Q 1/68; G01N 33/553

(52) U.S. Cl. 435/6; 436/526

(57) ABSTRACT

The present invention relates in general to the field of biotechnology. Particularly, the invention relates to a process for the controlled uptake and delivery of biomolecules onto solid, semi-solid or gel-like surfaces under the influence of magnetic fields.

### FIELD OF THE INVENTION

This application claims priority on German Patent Application No. 103 21 809.2, filed May 14, 2003, the entire disclosure of which is incorporated herein by reference.

The present invention relates generally to the field of biotechnology. Particularly, the invention relates to a method for the controlled uptake and delivery of biomolecules onto solid, semi-solid or gel-like surfaces under the influence of magnetic fields.

### BACKGROUND OF THE INVENTION

Since several decades, biomolecules, such as, in particular, nucleic acids have already been put onto carriers, in order that they or suitable binding partners can be investigated. While the process of controlled dosage uptake of biomolecules from supply vessels has been, for a long time, carried out by means of pipettes or comparable devices, the development of miniaturized systems and automated processes, in particular, led to the requirement, particularly with multiplex applications, such as the production of biomolecule arrays, that biomolecules be immobilized in a precise, grid-like arrangement, wherein smaller quantities of a desired, mostly aqueous probe must be placed in discreet positions on suitable surfaces. The reliability, standardizability and reproducibility of such systems leaves much to be desired, however.

On the one hand, buffer systems are used for printing of biomolecules, that are chemically reactive and can undergo desired reactions with the mostly functionalized surfaces. Because it is mostly desired to produce a covalent coupling of biomolecules to surfaces, so that they remain in their defined positions even after subsequent handling steps, the selection of a suitable printing buffer is of decisive significance for the reliability of the intended investigation results. With respect to so called biochips made of glass, for example, nucleic acid probes are often bound in a nucleophyllic reaction by means of amino reactive groups found on the chip. Because the most often used buffers also possess nucleophyllic properties, this can lead to a competitive inhibition of the anticipated coupling results, which in view of the high requirements of specificity and sensitivity of such systems, cannot be accepted. Furthermore, the dosaging properties of conventional composition cannot be said to be optimal because of a too small viscosity. Furthermore, particular biomolecules, such as, for example insulin, dissolve badly or not at all in buffers according to the state of the art, because of which a quantitative and qualitatively constant printing quality can often not be realized.

Devices that allow highly parallel applications, such as DNA arrays or micro arrays with other biomolecules are presently produced with needle printers or a type of ink jet printer. In this case, the medium in which the biomolecules are dissolved plays a decisive role with respect to the kind of dosaging, the dosing quantity, as well as the shape of the drops. Should differing biomolecules be printed on the same device, for which no common solvent is readily available, the person of skill in the art has a problem in that the differing solvents have differing surface properties with respect to the printed surface relating to the crosslink ability, contact angle, viscosity, drying rate and surface tension. In addition, in the case of the use of biomolecules that must be dissolved in aggressive solvents (for example, HCL, NaOH), this can lead to corrosion of the apparatuses or interference with the coupling chemistry (for example silane).

### SUMMARY OF THE INVENTION

The object of the present invention lies therefore in the provision of a method for depositing, or, as the case may be, printing, biomolecules on a surface, in which the above-described disadvantages of the state of the art are overcome.

The object is solved according to the present invention by the method according to the main claim. Particular embodiments of the method are represented in the dependent claims.

Furthermore, the present invention provides a method for analysis or diagnosis of analytes as well as a device for carrying out the process.

The above used terms “biological molecules” and “biomolecules” encompass all manner of substances and compounds substantially of biological origin, that have properties that are relevant in the framework of scientific and diagnostic and/or pharmaceutical applications. Encompassed are not only native molecules, such as may be isolated from natural sources, but also derivative forms, fragments and derivates, as well as recombinant forms and artificial molecules, as long as they comprise at least one property of the native molecule. Preferred biomolecules are such that can be applied for analytical, diagnostic and/or pharmaceutical purposes, such as nucleic acids and their derivatives (DNA, RNA, PNA, LNA, ribosomes, oligonucleotides, plasmids, chromosomes), peptides and proteins (enzymes, receptor proteins, protein complexes, peptide hormones, antibodies) as well as biologically-active fragments of the same, carbohydrates and their derivatives, such as, particularly, glycocilated proteins and glycosides, and fats, fatty acids and lipids.

It clear that the process according to the present invention can be applied to cellular tissues and complete cells as well as portions of the same (organelles, membranes, and membrane fragments, etc.), to the extent that the above are carriers of the above biomolecules. For this reason tissues, cells and portions of the same are fundamentally encompassed by the term “biomolecule”.

The method according to the present invention for printing onto a surface by the influence of magnetic fields, a preferably predetermined, defined number of biomolecules, covalently bound to a ferromagnetic particle by chemical, enzymatic and/or photochemically splittable crosslinkers, comprises the following steps:

Magnetic-field-controlled uptake of the preferably predetermined, defined number of particle bound biomolecules with a magnetic or magnetizable dosaging device, and

Delivery of the uptaken biomolecules onto the surface, whereby after splitting of the crosslinker, the remaining portion of the biomolecule comprises at least one functional group over which a covalent immobilization of the biomolecule onto the surface can take place.

According to a preferred embodiment, the adjustment of the field strength of the magnetic field by which the uptake of the defined number of biomolecules is controlled is carried out on the basis of standard values.

It has been determined according to the present invention that particularly disadvantages of systems in which substantially fluid probes or analyte preparations are printed can be overcome in that the uptake and delivery of biomolecules that are bound to ferromagnetic particles is assisted by help of magnetic fields.

Multifarious applications of ferromagnetic particles as carriers or carrier substances are known in the state of the art. For example, so-called “magnetic beads” from the company Dynal (Norway) are offered in various sizes and with various surface modifications which can be chosen according to application and applied to the present invention (see www.dynal.no, the entire content of this website is hereby incorporated by reference herein). Such magnetic particles comprise, for example, epoxy, amino, tosyl and/or carboxy groups and thereby allow the covalent conjugation of various biomolecules.

These ferromagnetic particles, which are themselves not permanently magnetic, are, in the state of the art, for example, attracted by means of commercially-available magnets and can, in this manner, be transported and fixed. In order to transport probes from one container (for example, a microtiter plate) to another container, transport is carried out through the use of rod-shaped electromagnets. These cannot, however, be utilized in a micro quantity application. The following proposed dosage device is, on the other hand, applicable for various size scales and allows the controlled printing of biomolecules onto surfaces in contact and non-contact processes.

It is clear to one of ordinary skill in the art with knowledge of the present invention that the reliability of the system according to the present invention with respect to the exact dosage ability depends determinably on control experiments, or, as the case may be, empirical data, because the relationship between applied field strength and the number (quantity) of the particles bound to the dosaging device is not known. Furthermore, one must determine the degree of accumulation of a particular particle with respect to the desired biomolecule. If one knows the number of biomolecules per particle as well as the relationship between the applied field strength and the bound particles, one can reliably determine the quantity of biomolecules taken up, and, thereby, exact dosaging can be reproducibly realized. According to the present invention, these parameters and variables are indicated as standard values. These standard values are either given or taken from data banks before carrying out the process.

### DETAILED DESCRIPTION OF THE ILLUSTRATIVE EMBODIMENTS

According to a preferred embodiment of the process according to the present invention, the field strength of the magnetic field of the dosaging device is decreased at the point and time of the delivery of the biomolecule. The magnetic field can therefore be turned off or adjusted to such a small field strength that the charged particles can leave the device.

According to a further preferred embodiment the biomolecules are selected from the group consisting of nucleic acids and their analogs, peptides, proteins, multi-protein complexes, saccharides, lipids and compounds, compositions and complexes comprising one or more of the above in combination.

The coupling of the biomolecule to the particle results, according to the present invention, by suitable crosslinkers, which are splittable either chemically, enzymatically and/or photochemically. The selection of suitable crosslinker molecules can be easily carried out by one of ordinary skill in the art with reference to the concrete task to be carried out. According to the present invention, crosslinkers are used, which, after splitting of the particles from the biomolecules, comprise, on a remaining portion, at least one functional group over which a covalent immobilization of the biomolecule onto the surface can result. Preferably, these functional groups are selected from group consisting of the functional groups benzophenone, thymidine, carbonic acid and amino groups.

According to a further preferred embodiment, the surface is a solid, semi-solid or gel-type surface. Furthermore, the surface is preferably a portion of an analytical or diagnostic device, such as, particularly, a microtiter plate, a test tube or a micro array.

According to an alternative embodiment, the surface to be printed can also have a magnetic field applied thereto in order to secure the deposition of particle-bound biomolecules to predetermined, defined positions on the same. This magnetic field ensures both positioning as well as a perfect printed result, because various particles bound to the dosaging device can be transferred in the desired position of the analytical device. It is in this case necessary that the field strength of the magnetic field of the surface or the individual field strengths of the magnetic fields of various surface regions are higher, at the point in time of the delivery of the biomolecules, than the field strength of the magnetic field of the dosaging device. So far as the magnetic field of the dosaging device is switched off for delivery, it is sufficient that a relatively small field strength of the surface or surface region is provided for realization of the advantages according to the present invention. Exact inputs for the necessary field strength can be delivered and applied as standard values (see above).

The method for analysis or diagnosis of analytes according to the present invention is based on the same invention principle and provides that the analytes covalently bound to ferromagnetic particles by means of chemically enzymatically and/or photochemically crosslinkers are taken up by means of a magnetic or magnetizable dosaging device and delivered onto a surface, whereby after splitting of the crosslinker the remaining portion of the analyte comprises at least one functional group over which the analyte can be covalently immobilized onto the surface.

According to a preferred embodiment, the surface is a portion of a microtiter plate, a test tube or a micro array. It is here particularly preferred that the surface has magnetic or magnetizable properties at least in the region of the position for analysis or diagnosis.

According to a further aspect of the present invention, magnetic or magnetizable dosage devices are provided with which the above-described processes can be carried out. Fundamentally, the selection of the suitable dosaging device according to the present invention is not limited and can preferably be selected from automated devices for printing.

According to the present invention, electromagnetic micropipettes are particularly suitable. Such systems comprise at least one ferromagnetic needle, which is preferably a portion of an electromagnet. According to a particularly preferred embodiment, the at least one needle is provided with an inert layer, such as, for example, Teflon, at least in a region with which it contacts the probe to be taken up, in order to minimize the fundamental reactivity of the needle. The electromagnet comprises, typically, a ferromagnetic core that is magnetized by a spool through which current flows. In order to avoid a permanent magnetization of the ferromagnetic core, the plurality of the electromagnet can be reversed in a fast sequence (for example 50 Hertz) continuously or at least directly after delivery of particles.

Furthermore, analytic or diagnostic devices for carrying out the process according to the present invention are provided that have magnetic or magnetizable properties at least in the region of positions provided for analysis or diagnosis.

With the foregoing embodiments as background, it is clear that the present invention also encompasses a system for printing a previously determined, defined number of biomolecules onto a surface under the influence of magnetic fields, as provided, comprising the following components:

Magnetic or magnetizable dosaging device for uptake of a defined number of particle bound biomolecules, and

Analytical or diagnostic device, whose surface comprises, at least in the region of the predetermined positions for analysis or diagnosis, magnetic or magnetizable properties, as well as biomolecules that are covalently bound to ferromagnetic particles by means of chemically, enzymatically and/or photochemically splittable crosslinkers, whereby after splitting of the crosslinker the remaining portion of the biomolecule comprises at least one functional group by means of which a covalent immobilization of the biomolecule on the surface can take place.

The invention will be further illustrated by means of the following example.

### EXAMPLE

Streptavidin coated magnetic beads from the company Dynal are modified with an oligonucleotide that has the following structure: biotin, 10T, EcoRI sequence, 10T, C₆ aminolinker. The amount of the built-in oligonucleotide can be determined by use of 1/100 of a fluorescence marked oligonucleotide without the EcoRI restriction site. A further oligonucleotide is hybridized to the above oligonucleotide so that the EcoRI sequence and the surrounding five nucleotides form a double strand which can be cut with the EcoRI enzyme. The carboxyterminus of an antibody is bound to these amino groups of the oligonucleotides by means of sulfo-NHS and EDC (Pierce). In this manner, magnetic beads with antibodies bound thereto result.

The beads are stored in a suitable buffer. A suitable buffers is, for example, PBS-buffer, pH 8. During use, the needle is inserted into the container with the beads and the current on the spool is turned on. The beads are attracted by the needle and are transported to the delivery position with the help of the needle. In highly parallel applications, the use of suitable pipette robots is suggested. As soon as the needle reaches its target position, the polarity of the magnetic field is switched for two seconds at 50 Hertz and thereafter turned off. Under these conditions, there results a printing, or as the case may be, delivery of various needle-bound beads. If desired, the magnetic field of the needle can also be reduced so that only a portion of the beads is delivered. A buffer is disposed at the target position of the printed surface in which the enzyme EcoRI is dissolved (suitable buffers are available with the enzyme). The DNA of the crosslinker is split in this buffer so that an antibody with a 10T end is released. The magnetic beads can then be further removed from the magnetic pipette to the extent desirable. The antibodies can then by bound by the 10T end to a 10A oligonucleotide fixed to the carrier. If the connection is to be irreversible, this can result from a suitable irradiation with UV light. At 260 nm, Thymidine radicals are produced that can covalently bind molecules (even DNA molecules) to the surface.

While the present invention has been described with reference to certain illustrative embodiments, one of ordinary skill in the art will recognize, that additions, deletions, substitutions and improvements can be made while remaining within the scope and spirit of the invention as defined by the appended claims.

What is claimed is:

1. A process for printing biomolecules, comprising the steps of:
providing a particle bound biomolecule that is covalently bound to a ferromagnetic particle over an chemically, enzymatically and/or photochemically splittable crosslinker;
controlling, with a magnetic field, an uptake of the particle bound biomolecule with a magnetic or magnetizable dosaging device;
delivering the uptaken biomolecule onto a surface;
splitting the crosslinker, whereby a the remaining portion of the biomolecule comprises at least one functional group; and
covalently immobilizing the biomolecule onto the surface with the at least one functional group.

2. A process according to claim 1, wherein said step of delivering further comprises delivering a predetermined number of biomolecules, and wherein said step of controlling comprises adjustment of the field strength of the magnetic field based on standard values.

3. A process according to claim 1, wherein a field strength of the magnetic field of the dosaging device is decreased at a point in time of the delivery of the biomolecules.

4. A process according to claim 1, wherein the biomolecule is selected from the group consisting of nucleic acids and their analogs, peptides, proteins, multi-protein complexes, saccharides, lipids and compounds, compositions and complexes of one or more of the above in combination.

5. A process according to claim 1, wherein the at least one functional group is selected from the group consisting of benzophenone, thymidine, carbonic acid and amino groups.

6. A process according to claim 1, wherein the surface is a solid, semi-solid or gel-like surface.

7. A process according to claim 6 wherein the surface comprises a portion of an analytical or diagnostic device.

8. A process according to claim 6, wherein the surface comprises a portion of a device selected from the group consisting of a microtiter plate, a test tube, and a micro array.

9. A process according to claim 1, wherein a magnetic field is applied to the surface in order to secure the delivery of the particle bound biomolecules to predetermined defined positions on the surface.

10. A process according to claim 9, wherein a field strength of the magnetic field of the surface is, at a point in time of delivery of the biomolecules, higher than a field strength of the magnetic field of the dosaging device.

11. A process for analysis or diagnosis of analytes, comprising the steps of:
providing an analyte covalently bound to a ferromagnetic magnetic particle by a chemically, enzymatically and/or photochemically splittable crosslinker;
taking up the analyte with a magnetic or magnetizable dosaging device;
delivering the analyte to a surface;
splitting the crosslinker, wherein a remaining portion of the analyte comprises at least one functional group; and
covalently immobilizing the remaining portion of the analyte onto the surface.

12. A process according to claim 11, wherein the surface is a portion of a microtiter plate, a test tube, or a micro array.

13. Process according to claim 10, wherein the surface comprises magnetic or magnetizable properties at least in a region of the predetermined positions for analysis or diagnosis.

14. A system for printing a predetermined, defined number of biomolecules onto a surface by influence of magnetic fields, comprising the following components:
a magnetic or magnetizable dosaging device for uptake of a defined number of particle bound biomolecules;
an analytical or diagnostic device, having a surface comprising magnetic or magnetizable properties at least in a region of predetermined positions for analysis or diagnosis; and
a biomolecule covalently bound to a ferromagnetic particle by chemically, enzymatically, and/or photochemically crosslinkers, wherein a remaining portion of the biomolecule after splitting of the crosslinker comprises at least one functional group covalently immobilizable onto the surface.

15. A process according to claim 2, wherein a field strength of the magnetic field of the dosaging device is decreased at a point in time of the delivery of the biomolecules

16. Process according to claim 11, wherein the surface comprises magnetic or magnetizable properties at least in a region of the predetermined positions for analysis or diagnosis.
